(19) United States
(12) Patent Application Publication
(10) Pub. No.: US 2015/0051496 A1
(43) Pub. Date: Feb. 19, 2015

(54) APPARATUS AND METHOD FOR CALCULATING A LOCATION OF AN OBJECT AND APPARATUS AND METHOD FOR FORMING AN OBJECT, USING AN ELECTROMAGNETIC WAVE IN A TERAHERTZ BAND

(71) Applicant: CANON KABUSHIKI KAISHA, Tokyo (JP)

(72) Inventor: Toshihiko Ouchi, Machida-shi (JP)

(21) Appl. No.: 14/384,360

(22) PCT Filed: Feb. 25, 2013
(86) PCT No.: PCT/JP2013/055763

§ 371 Date: Sep. 10, 2014

(30) Foreign Application Priority Data
Mar. 14, 2012 (JP) 2012-057251

(51) Int. Cl. A61B 5/00

(52) U.S. Cl. CPC A61B 5/0059
USPC 600/473

(57) ABSTRACT

An apparatus includes an emitter unit configured to irradiate an object under observation with a terahertz wave, a receiver unit configured to receive a reflected terahertz wave returning from the object under observation, and a data processing unit configured to calculate a propagation time period spent in a propagation of the terahertz wave from the emitter unit to the receiver unit based on a signal received by the receiver unit and calculate a location of an abnormal tissue in the object under observation based on the propagation time period.

### TECHNICAL FIELD

The present invention relates to an apparatus and a method for calculating a location of an abnormal tissue in an object under observation by using an electromagnetic wave in a terahertz band, and an apparatus and a method of forming an image of an object which may include an abnormal tissue under observation by using an electromagnetic wave in a terahertz band. More particularly, the present invention relates to an apparatus and a method of detecting a location of an abnormal tissue such as a cancer tissue on a surface of or in the inside of a living object, and an apparatus and a method of forming an image of an object which may include an abnormal tissue such as a cancer tissue on a surface of or in the inside of a living object under observation by using an electromagnetic wave in a terahertz band.

### BACKGROUND ART

In recent years, a nondestructive sensing technique has been developed that uses an electromagnetic wave in terahertz (THz) wave band (an electromagnetic wave with a frequency in a range from 30 GHz to 30 THz, which will be hereinafter referred to as a terahertz wave). The electromagnetic wave in this frequency band has been used in a wide variety of applications including an imaging technique for use in a see-through examination apparatus safer than a see-through examination apparatus using an X-ray, a spectroscopy technique for determining an absorbing spectrum or a complex dielectric constant of a substance thereby investigating a physical property thereof, a measurement technique for determining a physical property such as a carrier concentration, a mobility, an electric conductivity, or the like, a technique of analyzing a biological molecule, etc.

As an example of a technique of obtaining a see-through image of an object using a terahertz wave is a terahertz time-domain spectroscopy apparatus (hereinafter referred to as a THz-TDS apparatus) configured to generate a terahertz pulse by irradiating a semiconductor or the like with ultrashort pulse laser light (PTL 1). PTL 1 discloses a technique of obtaining an image of an object based on received signals of terahertz pulses passing through various spatially-different portions of the object.

However, in a case where the apparatus is used to detect an abnormal tissue on the surface of or in the inside of a living body, simple analysis of signals of electromagnetic pulses passing through various spatially-different parts is not sufficient to obtain an image of the abnormal tissue, but it is necessary to reconstruct the image taking into account signals scattered or reflected in the inside of the living body. PTL 2 discloses an apparatus configured such that a propagation time of a microwave after radiation from a microwave source is measured using a plurality of antennas and a plurality of receiver units, and an abnormal tissue is detected based on a difference in propagation time among signals received by different antennas and receiver units. In this apparatus, the radiation of the microwave is performed with reference to a reference clock, and periods of time elapsed from the radiation from a plurality of radiation units to the arrival at the receiver units are determined using a phase-locked circuit that operates in synchronization with the reference clock.

### CITATION LIST

### Patent Literature

PTL 1 U.S. Pat. No. 5,623,145

PTL 2 Japanese Patent Laid-Open No. 2010-69158

### Non Patent Literature

NPL 1 “Breast cancer detection using microwave imaging-Reduction of multiple reflection,” The 50th Annual Conference of Japan Society for Medical and Biological Engineering, O1-13-2.

NPL 2 Journal of Biomedical Optics 10 (6), 064021 (2005)

### SUMMARY OF INVENTION

### Technical Problem

In the apparatus disclosed in PTL 2, a microwave of 5 GHz (about 6 cm in wavelength) is employed. When such a microwave is used, the wavelength thereof is typically on the order of centimeters, which does not provide a sufficiently high resolution to detect early-stage cancer with a size on the order of millimeters. Furthermore, because of multipath caused by reflection in a living body, an error may occur in measurement of the propagation time, which may make it further difficult to achieve high detection accuracy (NPL 1).

On the other hand, if a terahertz wave with a frequency equal to or higher than 30 GHz (with a wavelength equal to or less than 1 cm) is used, a spatial resolution on the order of millimeters or a higher resolution is obtained. Furthermore, the microwave is absorbed greatly by water content in a living body, which results in a reduction in influence of multipath. For example, in a case of a skin, the absorption coefficient thereof is about 100 cm⁻¹ (NPL 2), and thus a great attenuation by a factor of about 5 e⁻⁵ per mm occurs.

However, in the terahertz imaging apparatus described above, no method or apparatus is disclosed for efficiently measuring propagation times of terahertz wave passing through a living body and reproducing an image based on the measured propagation times. Furthermore, it is difficult to construct a terahertz imaging apparatus by simply applying a technique employed in microwave imaging apparatuses, because a generation/detection unit used in the terahertz imaging apparatus is absolutely different from that employed in the microwave imaging apparatuses.

### Solution to Problem

According to an aspect, an apparatus includes an emitter unit configured to irradiate an object under observation with a terahertz wave, a receiver unit configured to receive a reflected terahertz wave returning from the object under observation, and a data processing unit configured to calculate a propagation time period spent in a propagation of the terahertz wave from the emitter unit to the receiver unit based on a signal received by the receiver unit and calculate a location of an abnormal tissue in the object under observation based on the propagation time period.

The apparatus using an electromagnetic wave in the terahertz band according to the aspect of the invention makes it possible to perform a high-resolution detection of a location of an abnormal tissue in an object under observation in a safer manner than in the case where an X-ray is used. Furthermore, use of a terahertz wave which attenuates greatly in a living body makes it possible to obtain a high-accuracy image without being influenced by noise caused by multiple reflections in the living body.

Further features of the present invention will become apparent from the following description of exemplary embodiments with reference to the attached drawings.

### BRIEF DESCRIPTION OF DRAWINGS

FIG. 1A is a diagram illustrating a whole configuration of an apparatus according to a first embodiment, and FIG. 1B is a cross-sectional view.

FIG. 2 is a diagram illustrating a structure of an emitter/receiver element according to the first embodiment.

FIGS. 3A and 3B are diagrams illustrating a signal detection according to the first embodiment.

FIG. 4 is a diagram illustrating a whole configuration of an apparatus and a structure of an emitter/receiver element according to a second embodiment.

FIG. 5 is a diagram illustrating an irradiation and receiving process using a probe according to a third embodiment.

FIG. 6 is a diagram illustrating an array of terahertz wave emitter/receiver elements according to a fourth embodiment.

### DESCRIPTION OF EMBODIMENTS

### First Embodiment

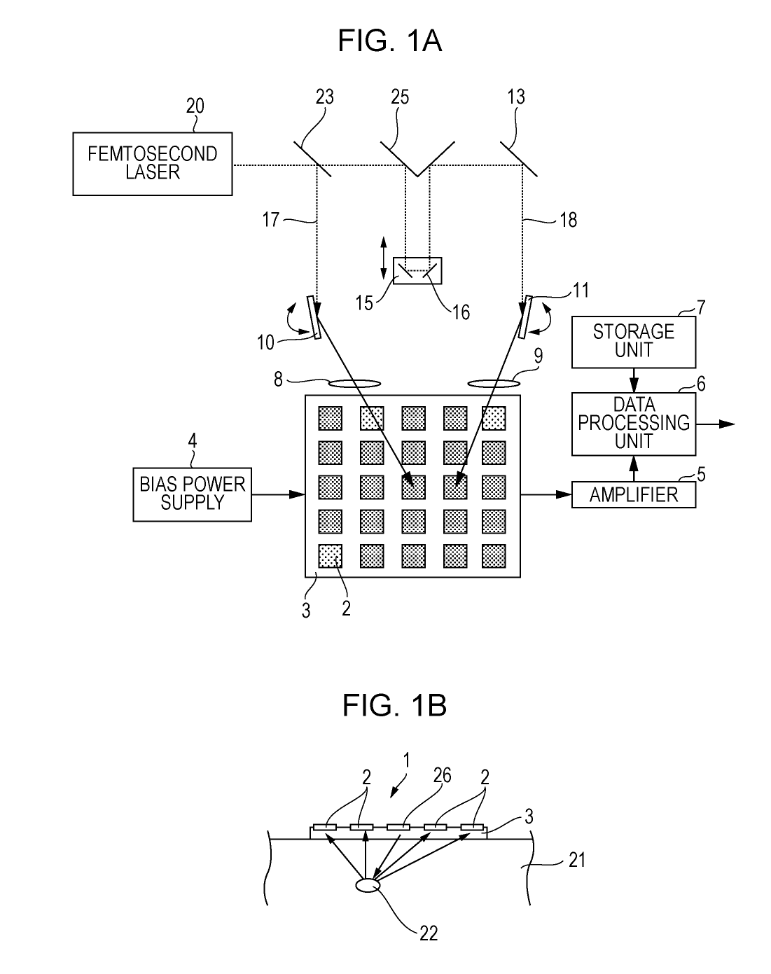

A first embodiment of the present invention is described below with reference to FIGS. 1A and 1B. An apparatus according to this embodiment is similar in configuration to a common THz-TDS apparatus except that a plurality of emitter/receiver elements are arranged in the form of an array, and each element is capable of providing both an emitting function and a receiving function by switching the functions in a time sharing manner. Each element may be used in different manners depending on whether it is used in both emitter and receiver, or it is used only in emitter or receiver.

Each element 2 in an emitter/receiver element array 1 may be configured to generate and receive a terahertz wave by irradiation of light. For example, a photoconductive element, a nonlinear crystal, etc. may be used as the element 2. In the case where a photoconductive element is used, an array of dipole antennas may be formed in an integrated manner on a substrate 3 such that each dipole antenna has a gap formed using a metal pattern on a surface of a photoconductive layer (with a typical thickness of 2 μm) such as a low-temperature (LT) grown GaAs layer. The substrate 3 may be formed using a material with a high transmittance in the terahertz wave band. Examples of materials usable for the substrate 3 includes a resin such as polyolefin cycloolefin, polyethylene, teflon (registered trademark), etc., a semiconductor such as diamond, quartz, sapphire, Si, GaAs, etc.

The photoconductive element may be produced, for example, by bonding a photoconductive layer using a pattern transfer technique, or by growing a photoconductive layer via a buffer layer or the like using an epitaxial growth technique. The thickness of the substrate is typically in a range from 0.3 to 1 mm. In a case where an object under observation is curved in shape, the substrate may be as thin as, for example, 100 μm such that the substrate is flexible. By forming the substrate to be flexible, it is possible to provide the emitter/receiver element array 1 placed on a curved surface.

The array structure may be formed, for example, such that elements 2 are arranged at equal intervals in the array including 5 rows and 5 columns as illustrated in FIG. 1A. Wirings (not illustrated) are provided such that a voltage supplied from a bias power supply 4 is applied to all elements. Furthermore, to make it possible to acquire a detection current from each element, wirings (not illustrated) are provided such that signals are input to an amplifier 5 in a state in which the bias voltage is turned off by a switch or such that an offset voltage is subtracted from each signal and a resultant value is input to the amplifier 5.

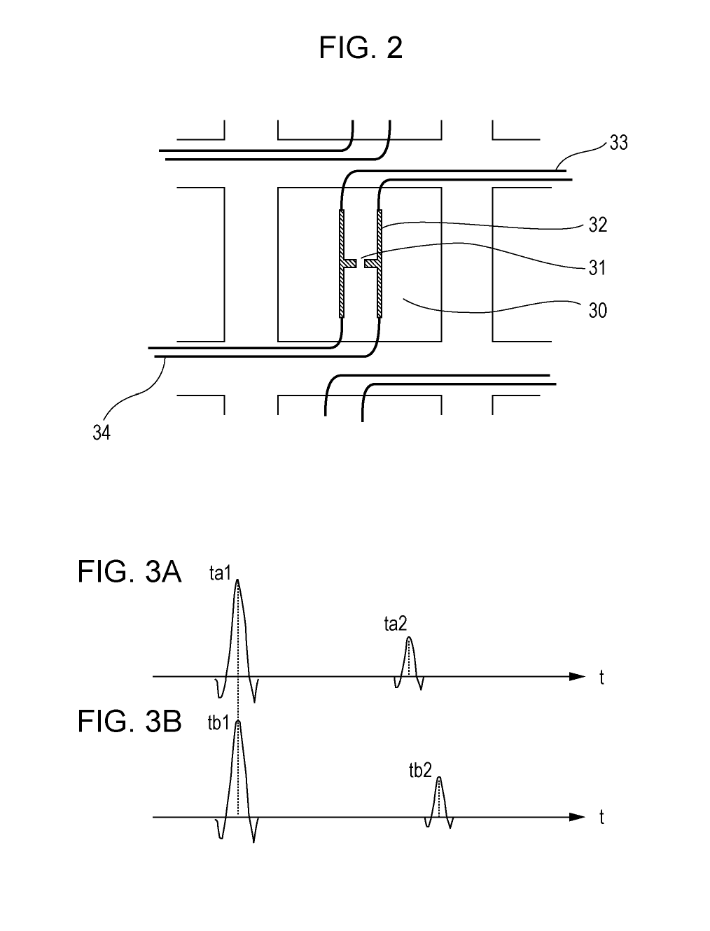

FIG. 2 is an enlarged view of an element formed using a photoconductive element. A dipole antenna 31 is formed on an LT-GaAs 30, and, to provide a bias voltage to the dipole antenna 21, strip lines 32 are formed which extend in parallel to each other. One of the strip lines 32 is connected to a voltage supply line 34, and the other one is connected to a detection line 33. To reduce a wiring area, wirings may be provided in a three-dimensional structure in which different wiring layers may be isolated from each other by an insulating film.

To provide a voltage via wirings in a time sharing manner, thin film transistors (not illustrated) may be integrated such that emitter/receiver elements are driven in a matrix manner.

In the example illustrated in FIGS. 1A and 1B, the elements 2 are separated from each other. Alternatively, one piece of non-separated LT-GaAs crystal may be used, and an insulating film having an array of windows may be formed over a wiring layer. To radiate or receive a terahertz wave using the array of elements, the elements may be irradiated with laser light serving as excitation light emitted from a femtosecond laser 20 while controlling an irradiation position using galvanomirrors 10 and 11 or the like, as illustrated in FIG. 1A.

For example, when a central element (located in a third row and a third column) is used as an emitter element, laser light is split into two beams using a half mirror 23 and one of the two laser beams, i.e., a laser light beam 17 (for irradiation) is directed onto a gap of the photoconductive element via the mirror 10 and a lens 8. When an adjacent element (located in a third row and fourth column, in the example illustrated in FIG. 1A) is used as a receiver element, a laser light beam 18 (for receiving) is passed through an optical delay unit including mirrors 25 and 16 and a driving unit 15 and further through mirrors 13 and 11 and a lens 9 such that the laser light beam 18 (for receiving) strikes the gap of the element. While irradiating the gap of the element, the optical delay unit is scanned and a waveform of a terahertz wave reflected from the object under observation is acquired via the amplifier 5 and a data processing unit 6.

In a case where light reflected from an abnormal tissue 22 in an object under observation 21 is detected at a plurality of locations for a single irradiation position as illustrated in FIG. 1B, the irradiation position of the emitter element may be fixed, and the galvanomirror 11 may be scanned such that a receiver element at a particular location is irradiated with the light, and a waveform of a terahertz wave from the element may be acquired. In the example illustrated in FIG. 1B, a terahertz wave is emitted from an element 26 located at a center as seen in cross section, and reflected light is received by four adjacent elements 2. The emission or the reception may be performed using a plurality of elements.

En element used as an emitter element may also be used as a receiver element as follows. If a propagation distance of a terahertz wave to be received is equal to or greater than a particular value, then irradiation timing of the excitation laser light irradiating the same element changes intermittently, and thus the element is capable of correctly functioning as the emitter element and the receiver element alternately. More specifically, a switching unit is provided in the apparatus to temporally switch the operation between the emitting operation and the receiving operation such that each element functions alternately as the emitter element and the receiver element. This makes it possible to realize the apparatus with a smaller number of elements. The laser light emitted from the femtosecond laser 20 used here may have a pulse width in the range from a few ten fs to 100 fs and a repetition frequency in the range from 10 MHz to 100 MHz (with a pulse-to-pulse interval in the range from 10 ns to 100 ns). For example, in a case where the distance from an emitter element to a location of an observation target is 0.5 mm (nearly equal to the thickness of the substrate), the propagation distance of the terahertz wave is twice the above-described distance because the terahertz wave goes forward and returns back to obtain a reflection image (the propagation distance is equal to 0.5 mm times 2, i.e., 1 mm), and the propagation time through free space is about 3 ps. In this case, when a terahertz pulse is generated at time t by irradiation with laser light beam 17, if the same point is irradiation with the laser light beam 18 about 2 ps later and if the delay stage is scanned for about a few ten ps, then it is possible to detect a reflected terahertz wave from an object under observation. That is, the illumination with the laser light beam 17 and the laser light beam 18 is performed repeatedly such that the laser light beam 17 and the laser light beam 18 are spaced apart in time in accordance with the repetition frequency, and terahertz waveforms are acquired. Hereinafter, this operation is referred to as a transceiver operation of the photoconductive element.

By repeating the above operation while changing the location of the terahertz emitter element irradiated with the laser light beam 17, it is possible to acquire a plurality of pieces of information on the propagation time from the emitter element to the receiver element at various locations.

FIGS. 3A and 3B illustrate examples of waveforms of terahertz waves which are emitted from one emitter element and received by different receiver elements. In each example, a first pulse is reflected at an interface between a substrate and an object under observation. In a case where the waveforms illustrated in FIGS. 3A and 3B are of signals received by different receiver elements located at equal distances from the emitter element, the first pulse signals may be detected at the same time, i.e., ta1 and tb1. Note that it is assumed that the substrate has no distortion, and the difference in propagation distance of the laser light beam 18 is corrected.

If there is no reflecting substance in the object under observation 21, second pulses are not observed. However, when there is an abnormal tissue 22 such as a cancer tissue, a refractive index difference causes the terahertz wave to be scattered, and scattered waves arrive at the respective receiver elements at different times. Therefore, second pulses are detected at different times, ta2 and tb2 as illustrated in FIGS. 3A and 3B unless the distance of the abnormal tissue from any receiver element is equal. Note that the difference in propagation time is measured as a difference in arrival time of the acquired pulses because THz-TDS is based on the principle of the time-domain measurement.

The propagation time elapsed from the illumination at the emitter element (emitter unit) to the reception at the receiver element (receiver unit) is calculated to acquire a plurality of pieces of propagation information, and then, based on the acquired propagation information, a 3-dimensional location of the abnormal tissue 22 in the object under observation is calculated. Note that the calculation is performed by the data processing unit 6. Alternatively, the calculation may be performed by software installed on a personal computer. In a case where only one piece of information of propagation time is available, the information may be combined with information of a location of the element and also (or instead) a location of the object under observation, to make it possible to calculate the location of the abnormal tissue. That is, it is possible to noninvasively obtain a 3-dimensional image of the object under observation including the inner image thereof. In a case where the object under observation is a living object, presence of water in the living object causes the terahertz wave to attenuate, and thus the observable range in a depth direction is typically 5 mm or less. In other words, an influence of multipath due to multiple reflection in the inside of the living object is reduced to a negligible level.

The spatial resolution in detecting an abnormal tissue is basically dependent on an element-to-element pitch. The length of a bias line of each photoconductive element may be set to about 3 mm or greater to avoid signal interference (multiple reflection in the element). The signal interference depends on a low-frequency bandwidth, and thus if the bias line length is equal to or greater than 3 mm, substantially no influence occurs at frequencies equal to or higher than 100 GHz. Therefore, in the array of elements according to the present embodiment, the element-to-element pitch is set to 3 mm.

To improve the resolution without being influenced by multiple reflection, the location of the element array may be moved stepwise such that the relative location of the element array with respect to the object under observation is moved a particular distance, for example, 1 mm in each step, and a signal is acquired at each location of the element array.

In a case where the substrate is flexible and thus the substrate is bent according to the shape of the object under observation, the degree of bending is detected at a location corresponding to the first pulse, so that the propagation time difference is allowed to be corrected based on the detected degree of bending. In this case, the apparatus may include a bending unit to physically bend the substrate. Furthermore, the apparatus may include a signal processing unit for handling bending configured to calculate the degree of bending based on the information on the propagation time, an incidence angle, etc., and process the signal based on the degree of bending.

Because the data acquired in during the process is huge in amount, it may take a very long time to analyze the data. To reduce the detection time, candidates for the type of the abnormal tissue and candidates for the location of the abnormal tissue in the living body, and predicted relationships between the signal and the candidates for the type and location may be described in a database and stored in a storage unit 7. The data processing unit 6 may compare the signal with the data in the database, which allows an increase in detection speed.

### EXAMPLE 1

In this first example, a 1.5 μm band fiber-type femtosecond laser is used as an excitation laser source 20. A sinusoidal voltage of 40 Vp-p is applied to a photoconductive element, and the photoconductive element is irradiated with ultrashort pulse light functioning as pumping light with an average power of 20 mW and with a pulse width of 30 fsec. A photoconductive element on a detection side is irradiated with probing light of 5 mW, and a detected current is converted into a voltage signal by a transimpedance amplifier with a gain of about 10⁷. A filter may be inserted as required. In a typical case, terahertz pulse with a peak of about 100 mV is observed using a lockin amplifier or the like. By modulating the optical path length in the probing path using a delay stage 15, it is possible to measure a time-domain waveform of the terahertz pulse irradiating the object under observation using a sampling technique. The acquired time-domain waveform is then Fourier-converted to obtain a frequency-domain signal with a bandwidth of 5 THz or greater.

In FIG. 1, the data processing unit controls the lockin amplifier and processes the signal output from the lockin amplifier by using a computer. The output signal is displayed on a display and stored as electronic data in the storage unit. Alternatively, the data may be stored in an external storage apparatus in a personal computer or a server.

The driving condition described above is merely an example, and the voltage and the illumination light power are not limited to the values described above. Furthermore, the excitation light source described above is merely an example, and other excitation light sources or other irradiation conditions may be employed.

In a case where a nonlinear crystal is used in the terahertz emitter/receiver element, it is not allowed to apply a sinusoidal wave bias voltage. Instead, in this case, a synchronous detection using an optical chopper may be employed.

In a case where the signal intensity is high enough, the synchronous detection may be unnecessary.

### Second Embodiment

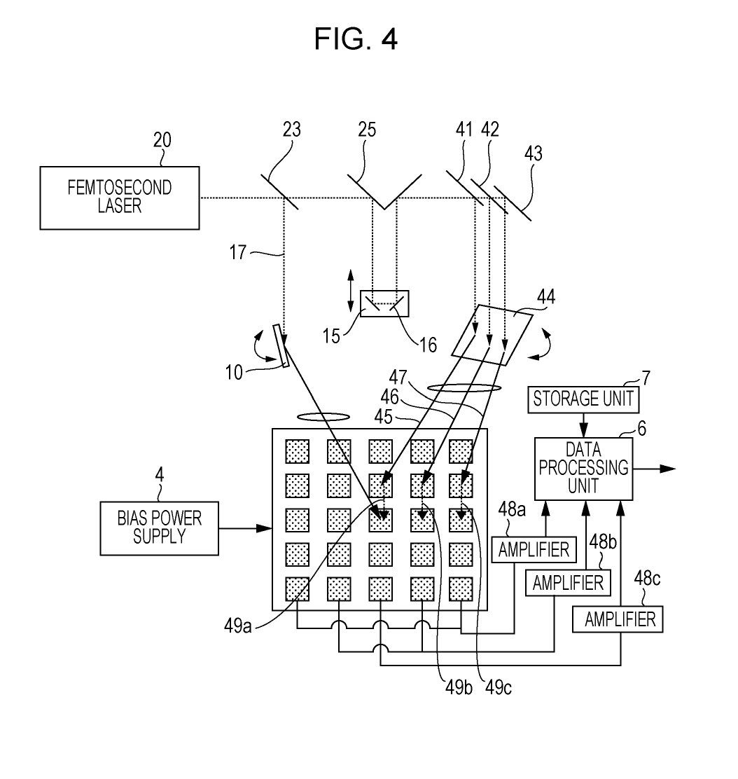

In a second embodiment described below, a plurality of receiver elements are driven simultaneously to receive a plurality of signals at a high rate. As illustrated in FIG. 4, after laser light beam is passed through an optical delay unit 15, the laser light beam is split into three beams 45 to 47 by beam splitters 41 and 42 and a reflecting mirror 43, and the three beams are directed by a single galvanomirror 44 toward elements serving as receivers. Instead of the single galvanomirror, a multufaceted deformable mirror or the like may be used to scan the respective beams in an independent and variable manner.

The locations of the beam splitters and the reflecting mirrors (41 to 43) are set such that the three laser beams (45 to 47) reach the receiver elements at different times. In this configuration, receiving signals from the three receiver elements are independently sent to three amplifiers (48a to 48c) via separate wirings, and terahertz time-domain waveforms of the signals are acquired by the data processing unit 6 and the storage unit 7 in a similar manner to the first embodiment.

In a case where the three elements are simultaneously irradiated with laser light provided in a single emitting operation as illustrated in FIG. 4, the amplifiers are configured to operate separately, and elements located in the same column share the same wirings. In this case, the three probing light beams are moved as indicated by three arrows 49a to 49c in a next step as illustrated in FIG. 4. More specifically, for example, the probing light beams are moved from an element in 2nd row and 3rd column to an element in 3rd row and 3rd column, from an element in 2nd row and 4th column to an element in 3rd row and 4th column, and so on. After elements located in a right-hand area are sequentially scanned, elements located in a left-hand area are sequentially scanned thereby acquiring signals from all pixels. By simultaneously acquiring three signals in a particular scanning area using the single optical delay unit 15 as described above, it is possible to acquire data at a higher speed than in the first embodiment.

In the present embodiment, it is assumed by way of example that three laser beams are used in simultaneous irradiation. However, the number of laser beams is not limited to three, but, a properly selected number of laser beams may be used. For example, five laser beams may be used, or as many laser beams as there are elements may be used. The respective elements may be configured in a similar manner to the first embodiment, and the laser may be driven in a similar manner to the first embodiment.

### Third Embodiment

In a third embodiment of the invention, the above-described irradiation method according to the second embodiment is extended such that a high-power femtosecond laser with a large beam diameter of about 20 mm is used to simultaneously irradiate all 5×5 elements, i.e., so as to irradiate a whole array of emitter/receiver elements 1 integrated on a substrate.

The elements are connected by a matrix wiring system using MOS switches such that one element is selected at each timing point at which the element is used as an emitter element or a receiver element and a voltage is applied to the selected element and a current is detected.

The timing of intermittently irradiating emitter/receiver elements with light and the transceiver operation may be performed in a similar manner to the first embodiment.

In the present embodiment, use of a high-power laser light source provides a merit that it becomes unnecessary to perform a high-precision control of an irradiation position using a galvanomirror.

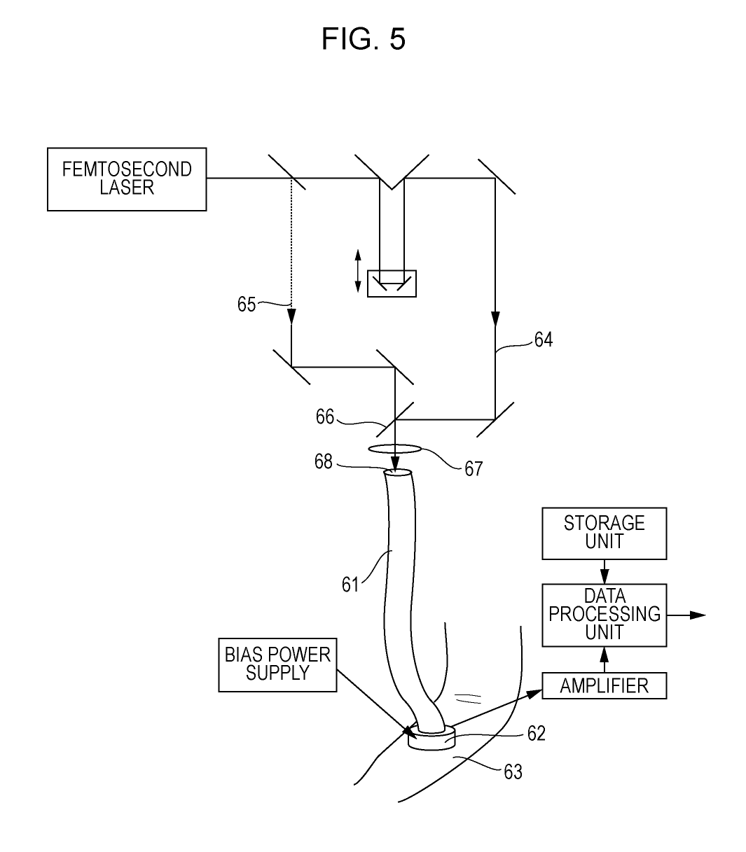

In the irradiation of the whole area, a spatial irradiation system may be used, or alternatively an irradiation system using a probe such as that illustrated in FIG. 5 may be used. In FIG. 5, reference numerals of parts similar to those in the previous figures are not shown. A generation laser light beam 65 and a detection laser light beam 64 are combined together by a half mirror 66 and entered to an optical fiber 61 via a lens 67. The resultant laser light beam propagates through the optical fiber 61 and illuminates the whole area of the emitter/receiver element array 1 such as that illustrated in FIGS. 1A and 1B disposed on the end 62 of the probe so as to drive the emitter/receiver element array 1 in the transceiver manner.

Although connections to a bias power supply, an amplifier, etc., for use in the operation are illustrated in a schematic manner in FIG. 5, wirings for the connections may be provided along the wall of the fiber 61 and the connections may be made at locations close to an input end 68 of the fiber 61. Although a part from the femtosecond laser to the input end 68 of the fiber 61 is formed by a spatial system in FIG. 5, this part may be configured in the form of a module.

In the example illustrated in FIG. 5, the object under observation is a person and the probe is brought into contact with an antebrachial region of the person. The abnormal tissue may be an abnormal or ill part of a tissue in a living body or a part subjected to surgery. Examples of abnormal tissues include a cancer on a surface of or below a skin of the antebrachial region, a burn part, a cured part after a transplant (surgery), etc. Further examples are an osteoporotic bone part, a swelling of a liver or a lien, a cirrhosis of a liver, etc. An abnormal tissue may occur not only in the antebrachial region but in other parts such as a breast, a joint, a head, etc.

It is also possible to bring the apparatus into contact with an internal organ exposed during a surgery operation, calculate a location of an abnormal tissue, and form an image based on the calculated location information thereby making it possible to visually determining the location of the abnormal tissue. Note that the probe may be used as an endoscope.

### Fourth Embodiment

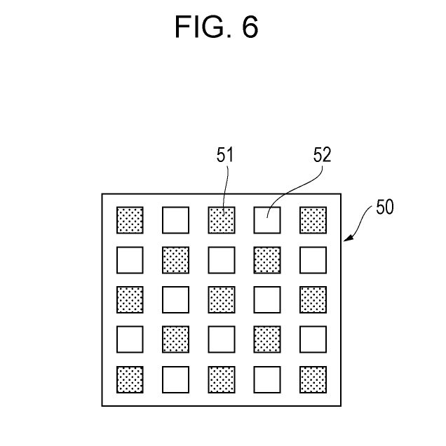

In a fourth embodiment described below, instead of a THz-TDS system, terahertz oscillators (or emitter element) or detectors (or receiver elements) are integrated in the form of an array. For example, an array of elements 50 is formed by arranging alternately oscillators 51 and detectors 52 at equal intervals (for example, at intervals of 2 mm) as illustrated in FIG. 6.

In a case where wirings are provided to separately drive the oscillators 51 and the detectors 52, when a terahertz wave is output from one oscillator, data is acquired using all detectors and locations of internal reflection points are analyzed via an image reconstruction process. In a case where there are a large number of pixels, driving may be performed in a matrix manner using a switching unit including integrated switch elements.

The elements may be of an electrically-driven type. For example, a resonant tunneling diode oscillator may be used as each oscillator, and a Schottky barrier oscillator may be used as each detector. These devices are advantageous in that they operate at room temperature. The oscillators may be of a plasma type, a quantum-cascade laser type, or the like, and the detectors may be of a multiple quantum well type, a thermal type, or the like.

Driving may be performed, for example, such that the oscillators are driven by pulses and the distance to a tissue of interest may be determined based on a difference in time between a signal propagating through the inside of the substrate and a signal reflected from an abnormal tissue in the living body.

### Other Embodiments

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-057251, filed Mar. 14, 2012, which is hereby incorporated by reference herein in its entirety.

### REFERENCE SIGNS LIST

1 emitter/receiver element array
2 element
6 data processing unit
21 object under observation
22 abnormal tissue

1. An apparatus comprising:
a plurality of elements disposed in an array, each of the plurality of elements being configured to emit or receive a terahertz wave;
a switching unit configured to temporally switch whether to make each of the plurality of elements operate as an emitter unit configured to irradiate an object under observation with a terahertz wave, or as a receiver unit configured to receive a reflected terahertz wave returning from the object under observation; and
a data processing unit configured to calculate a propagation time period spent in a propagation of the terahertz wave from the emitter unit to the receiver unit based on a signal received by the receiver unit and calculate a location of an abnormal tissue in the object under observation based on the propagation time period,
wherein the plurality of elements includes a first element and a plurality of second elements disposed around the first element, and
wherein the switching unit makes the first element operate as the emitter unit and makes each of the plurality of second elements operate as the receiver unit.

2. (canceled)

3. The apparatus according to claim 2, wherein
the emitter unit or the receiver unit includes a photoconductive element, and
the switching unit changes an irradiation position at which the photoconductive element is irradiated with the laser light.

4. The apparatus according to claim 2, wherein the switching unit drives elements of an electrically-driven type separately or in a matrix manner.

5. The apparatus according to claim 1, wherein the emitter unit and the receiver unit are integrated on the same substrate.

6. The apparatus according to claim 5, wherein the substrate functions as an end part of a probe configured to be allowed to be brought into contact with the object under observation.

7. The apparatus according to claim 1, further comprising a storage unit configured to store data acquired in advance in terms of a relationship between a type of the abnormal tissue in the object under observation and a received signal, for use in calculating a location of the abnormal tissue.

8. The apparatus according to claim 1, wherein
the object under observation is a living body, and
the abnormal tissue is a cancer tissue.

9. (canceled)

10. (canceled)
